(19) United States
(12) Patent Application Publication
(10) Pub. No.: US 2015/0291591 A1
(43) Pub. Date: Oct. 15, 2015

(54) PROCESS FOR THE PREPARATION OF POLYMORPHS OF DORIPENEM

(71) Applicant: RANBAXY LABORATORIES LIMITED, New Delhi, Delhi (IN)

(72) Inventors: Durga Shankar, Bundi (IN); Vishwesh Pravinchandra Pandya, Jamnagar (IN); Hashim Nizar Poovanathil Nagoor Meeran, Pathanamthitta (IN); Shailendra Kumar Singh, Gurgaon (IN); Mohan Prasad, Gurgaon (IN); Sudershan Kumar Arora, Gurgaon (IN)

(21) Appl. No.: 14/357,129

(22) PCT Filed: Nov. 5, 2012
(86) PCT No.: PCT/IB2012/056169

§ 371 Date: May 8, 2014

(30) Foreign Application Priority Data
Nov. 8, 2011 (IN) 3167/DEL/2011

(51) Int. Cl. C07D 477/06

(52) U.S. Cl. CPC C07D 477/06; C07B 2200/13

(57) ABSTRACT

The present invention relates to a process for the preparation of polymorphs of doripenem.

### FIELD OF THE INVENTION

The present invention relates to a process for the preparation of polymorphs of doripenem.

### BACKGROUND OF THE INVENTION

Doripenem is a synthetic broad-spectrum carbapenem antibiotic and commercially available as the monohydrate. Doripenem is chemically known as (4R,5S,6S)-3-[[(3S,5S)-5-[[(Aminosulfonyl)amino]methyl]-3-pyrrolidinyl]thio]-6-[(1R)-1-hydroxyethyl]-4-methyl-7-oxo-1-azabicyclo[3.2.0]hept-2-ene-2-carboxylic acid, (Formula I).

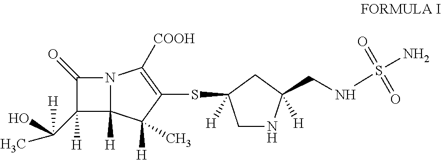 [SMILES: C[C@@H](O)[C@H]1C(=O)N2C(C(=O)O)=C(S[C@@H]3CN[C@H](CNS(N)(=O)=O)C3)[C@H](C)[C@H]12]

Doripenem is an effective antibiotic agent for the treatment of complicated intra-abdominal infections caused by Escherichia coli, Klebsiella pneumoniae, Pseudomonas aeruginosa, Bacteroides caccae, Bacteroides fragilis, Bacteroides thetaiotaomicron, Bacteroides uniformis, Bacteroides vulgatus, Streptococcus intermedius, Streptococcus constellatus and Peptostreptococcus micros.

Processes for preparation of polymorphs of doripenem are described in U.S. Pat. No. 6,111,098 and U.S. Publication No. 2008/0207586.

Yutaka Nishino et al., Org. Proc. Res. Dev., 7(6), p. 846-850 (2003), describes a process for preparing a non-sterile crystal of doripenem by adding methanol and seed crystal to the aqueous solution of doripenem.

### SUMMARY OF THE INVENTION

The present inventors have developed a simple and advantageous process for the preparation of the Type I crystal and the Type IV crystal of doripenem. By employing the present method, Type I and Type IV crystals of doripenem can be prepared in a reproducible and stable manner. The Type I and Type IV crystals obtained by the present invention are suitable for the development of pharmaceutical dosage forms.

### DETAILED DESCRIPTION OF THE INVENTION

A first aspect of the present invention provides a process for the preparation of Type I crystalline form of doripenem having interplanar spacing (d) values measured by XRPD substantially at 11.97, 5.99, 5.33, 4.42, 4.34, 4.21, 3.2, 2.99 and 2.71±0.02, wherein the process comprises:

a) providing an aqueous solution of doripenem;

b) treating the solution obtained in step a) with methanol;

c) seeding the mixture obtained from step b) with Type I or Type IV crystals; and

d) isolating the Type I crystal of doripenem.

The starting doripenem may be prepared according to the methods described in the prior art, for example, U.S. Pat. Nos. 6,111,098 and 5,317,016 and PCT Publication Nos. WO 2009/118680, WO 2007/029084 or WO 2006/117763. The aqueous solution of doripenem may be prepared by dissolving doripenem in water, or directly obtained, for example, by layer separation from the reaction mixture in which doripenem is formed. The temperature of the aqueous solution of doripenem may be maintained at about −20° C. to about 10° C., for example, about 0° C. to about 5° C. The aqueous solution of doripenem is treated with methanol. The treatment with methanol may be carried out by adding methanol to the aqueous solution of doripenem or by adding the aqueous solution of doripenem to methanol. The treatment with methanol may be carried out over a period of about 1 minute to about 10 hours, for example, about 4 minutes to about 15 minutes. The mixture so obtained is seeded with Type I or Type IV crystals of doripenem. The mixture may be stirred at a temperature of about 10° C. to about −30° C., for example, about −3° C. to about −15° C. The mixture may be stirred for about 0.5 hours to about 24 hours, for example, for about 2 hours to about 4 hours. The mixture so obtained may optionally be further treated with methanol. The further treatment with methanol may be carried out over a period of about 5 minutes to about 10 hours, for example, about 15 minutes to about 1 hour. The mixture may be stirred further at a temperature of about 10° C. to about −30° C., for example, −3° C. to about −15° C., for about 0.5 hours to about 24 hours, for example, about 2 hours to about 4 hours. The Type I crystal of doripenem is isolated from the mixture so obtained. The isolation may be carried out, for example, by filtration and/or decantation. The Type I crystal of doripenem so obtained may be further dried under vacuum at a temperature of about 40° C. to about 55° C., for example, about 45° C. to about 50° C. The Type I crystal of doripenem, so obtained, has interplanar spacing (d) values in XRPD substantially at 11.97, 5.99, 5.33, 4.42, 4.34, 4.21, 3.2, 2.99 and 2.71±0.02.

A second aspect of the present invention provides a process for the preparation of a Type IV crystalline form of doripenem having interplanar spacing (d) values measured by XRPD substantially at 6.80, 5.92, 5.59, 5.34, 4.31, 4.23, 4.01, 3.73, 3.42, 3.17, 3.08 and 2.83±0.02, wherein the process comprises:

a) dissolving doripenem in water at a temperature of about 15° C. or above;

b) cooling the solution obtained in step a) to 10° C. or below; and

c) isolating a Type IV crystalline form of doripenem

wherein step a) to step c) do not involve seeding.

The starting doripenem may be prepared according to the methods provided in the prior art, for example, U.S. Pat. No. 5,317,016 and PCT Publication Nos. WO 2009/118680 or WO 2006/117763. The doripenem is dissolved in water at a temperature of about 15° C. or above, for example, about 15° C. to about 55° C. The aqueous solution of doripenem is cooled to a temperature of about 10° C. or below, for example, about 10° C. to about −10° C. The mixture so obtained may be stirred for about 30 minutes to about 20 hours, for example, about 1 hour to about 15 hours. The solution may be further cooled to a temperature of about 10° C. or below, for example, about 10° C. to about −10° C. The mixture so obtained may be stirred for about 30 minutes to about 20 hours, for example, about 10 hours to about 15 hours. Seeding with any crystals of doripenem and/or the addition of any other solvent is not needed. The Type IV crystalline form of doripenem is isolated from the mixture, for example, by filtration, solvent removal, decantation, or a combination thereof. The Type IV crystalline form of doripenem may optionally be washed with a C₃-C₇ ketone, for example, acetone. The Type IV crystalline form of doripenem, so obtained, has interplanar spacing (d) values in XRPD substantially at 6.80, 5.92, 5.59, 5.34, 4.31, 4.23, 4.01, 3.73, 3.42, 3.17, 3.08 and 2.83±0.02.

### DETAILS OF THE FIGURES

FIG. 1 depicts the XRPD of the Type I crystalline form of doripenem.

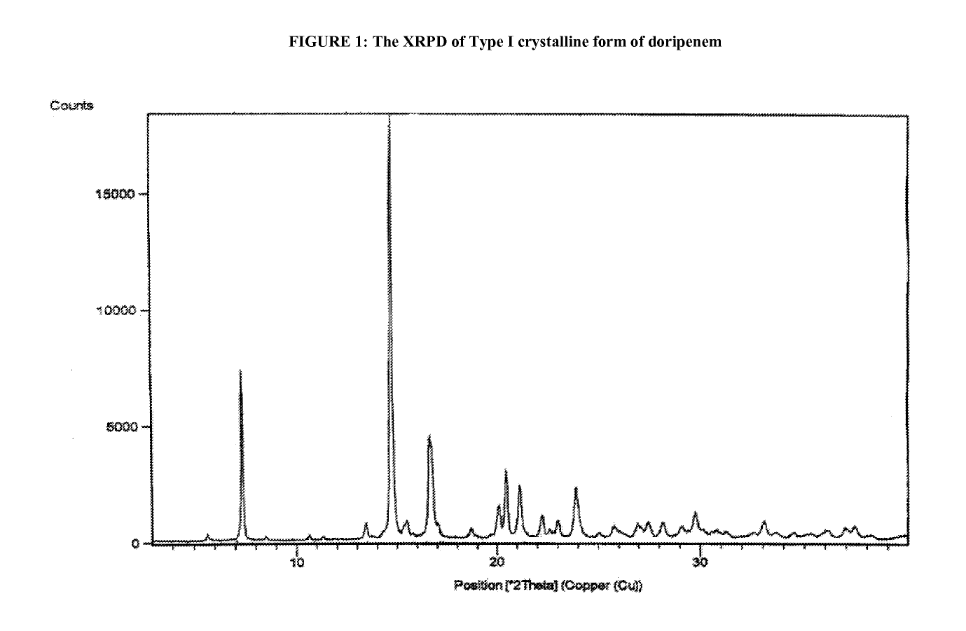

FIG. 1A provides the table of values for the XRPD of FIG. 1.

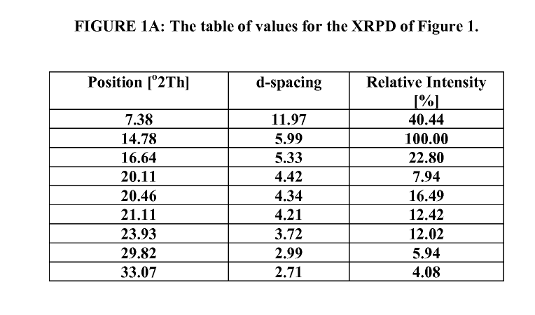

FIG. 2 depicts the XRPD of the Type IV crystalline form of doripenem.

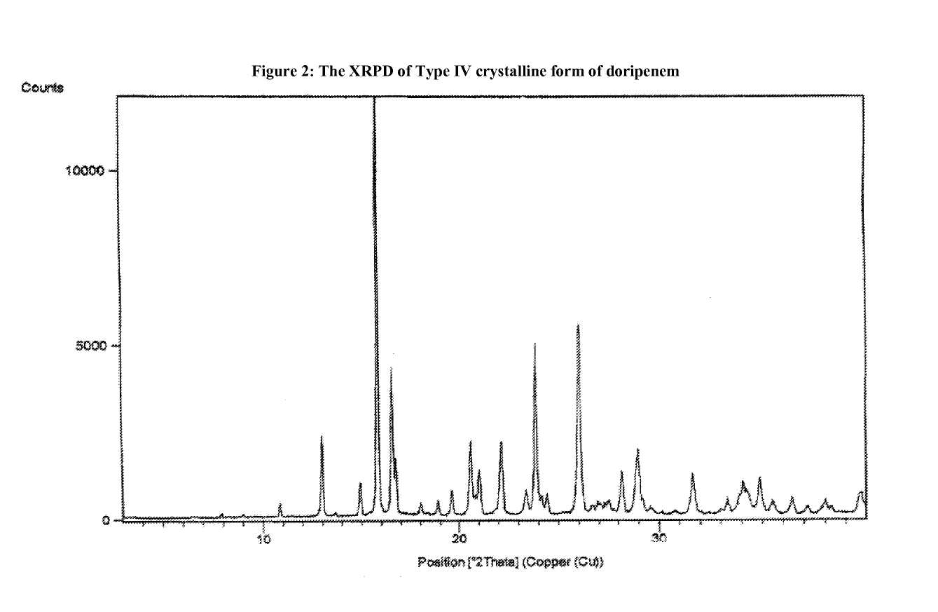

FIG. 2A provides the table of values for the XRPD of FIG. 2.

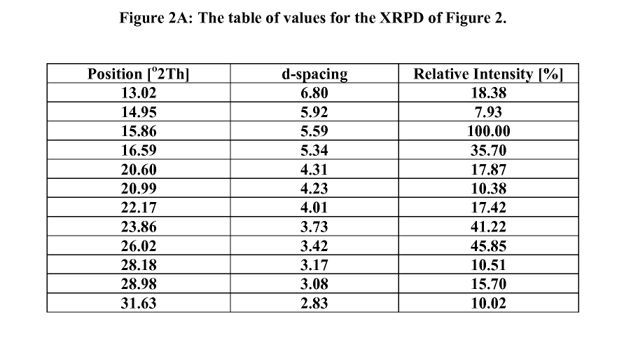

XRPD of the samples were determined using an X-Ray diffractometer, Rigaku Corporation, RU-H3R, Goniometer CN2155A3, X-Ray tube with Cu target anode, Power: 40 KV, 100 Ma, Scanning speed: 2 deg/min step: 0.02 deg, Wave length: 1.5406 A.

While the present invention has been described in terms of its specific embodiments, certain modifications and equivalents will be apparent to those skilled in the art and are intended to be included within the scope of the present invention.

### EXAMPLES

### Example 1

### Preparation of the Type IV Crystalline Form of Doripenem

Amorphous doripenem (100 g) was dissolved in distilled water (160 mL) at 15° C. in 30 minutes and a clear solution was obtained. The mixture was cooled to 10° C. and stirred for 1 hour. The mixture was further cooled to 0° C., stirred for 12 hours, filtered and washed with acetone (2×100 mL) and the wet solid was dried at 45° C. to 50° C. under vacuum to obtain the title product.
Yield: 50 g
Purity: 99.87%

### Example 2

### Preparation of the Type I Crystalline Form of Doripenem Using Type IV Seed Crystal

4-Nitrobenzyl-4-(acetylsulfanyl)-2-{[(tert-butoxycarbonyl)(sulfamoyl)amino]methyl}pyrrolidine-1-carboxylate (279.9 g) and acetyl chloride (42.96 g) were dissolved in methanol (1250 mL) and the mixture was refluxed for 3 hours. The mixture was cooled to 25° C. The reaction mixture was poured into a mixture of methylene chloride (1500 mL) and de-ionized water (2500 mL). The organic layer was separated, washed with de-ionized water (1250 mL) and concentrated under reduced pressure. The concentrate so obtained was dissolved in N,N-dimethylformamide (250 mL) and added to 4-nitrobenzyl (4R)-3-[(diphenoxyphosphoryl)oxy]-6-[(1R) -1-hydroxyethyl]-4-methyl-7-oxo-1-azabicyclo[3.2.0]hept-2-ene-2-carboxylate (250 g) in N, N-dimethylformamide (1000 mL) at 0° C. to 5° C. Diisopropylethylamine (76.14 g) was added dropwise to the reaction mixture at −35° C. to −25° C. The reaction mixture was stirred for 2 to 4 hours at −30° C. to −25° C. The reaction mixture was poured into a mixture of ethyl acetate (3000 mL) and de-ionized water (2500 mL). The organic layer was separated. The aqueous layer was further extracted with ethyl acetate (1750 mL). The combined organic layers were washed with 5% aqueous sodium chloride solution (2×875 mL). 2.5% Palladium on carbon in an aqueous buffer (1750 mL) containing N-methylmorpholine (42.51 g) and acetic acid (25.25 g, pH 5.8 to 6.5) was added to the organic layer. The biphasic reaction mixture was hydrogenated for 2 hours to 3 hours under 4 to 6 kg/cm² pressure at 10° C. to 25° C. The reaction mixture was filtered and washed with a mixture of ethyl acetate (1250 mL) and de-ionized water (500 mL). The aqueous layer (2250 mL) was separated and degassed under reduced pressure while simultaneously cooled to 0° C. to 5° C. Methanol (1250 mL) was added to the aqueous solution at 0° C. to 5° C. over a period of 5 to 10 minutes. The solution was seeded with Type IV seeds (12.5 g) obtained according to Example 1. The reaction mixture was stirred for 3 hours at −5° C. to −10° C. and methanol (750 mL) was added over a period of 45 minutes to 60 minutes at −5° C. to −10° C. The mixture was stirred for 3 hours at −5° C. to −10° C. The reaction mixture was filtered and washed with a cold mixture (0° C. to −5° C.) of methanol (175 mL) and de-ionized water (75 mL) followed by acetone (500 mL). The wet solid was dried at 45° C. to 50° C. under vacuum to yield the title product.
Yield: 80 g
Purity: 99.92%

### Example 3

### Preparation of the Type I Crystalline form of Doripenem Using Type I Seed Crystal

Example 2 was repeated by using Type I seed crystals to obtain the title product. The Type I seed crystals were prepared according to Example 2.
Yield: 80 g
Purity: 99.92%

### Comparative Example

### Preparation of Doripenem (Type I crystal) Without the Use of Seed Crystal

Example 2 was repeated without using any seed crystal and no precipitation was observed, even after 7 hours of stirring.

We claim:

1. A process for the preparation of a Type I crystalline form of doripenem having interplanar spacing (d) values measured by XRPD substantially at 11.97, 5.99, 5.33, 4.42, 4.34, 4.21, 3.2, 2.99 and 2.71±0.02, wherein the process comprises:
a) providing an aqueous solution of doripenem;
b) treating the solution obtained in step a) with methanol;
c) seeding the mixture obtained from step b) with Type I or Type IV crystals; and
d) isolating a Type I crystalline form of doripenem.

2. The process of claim 1, wherein the temperature of the solution at step b) is about a −20° C. to about 10° C.

3. The process of claim 1, further comprising:
e) further treating the Type I crystalline form of doripenem with methanol.

4. The process of claim 1, wherein step b) is carried out for about 1 minute to about 10 hours.

5. A process for the preparation of a Type IV crystalline form of doripenem having interplanar spacing (d) values in XRPD substantially at 6.80, 5.92, 5.59, 5.34, 4.31, 4.23, 4.01, 3.73, 3.42, 3.17, 3.08 and 2.83±0.02, wherein the process comprises:
a) dissolving doripenem in water at a temperature of about 15° C. or above;
b) cooling the solution obtained in step a) to 10° C. or below; and
c) isolating Type IV crystalline form of doripenem.
wherein steps a) to c) does not involve seeding.

6. The process of claim 4, wherein step b) is carried out at a temperature of about 10° C. to about −10° C.

7. The process of claim 5, further comprising:
d) the Type IV crystalline form is further washed with a C₃-C₄ ketone.
